(19) United States
(12) Patent Application Publication
(10) Pub. No.: US 2024/0233939 A9
(43) Pub. Date: Jul. 11, 2024

(54) STROKE DIAGNOSIS AND THERAPY ASSISTANCE SYSTEM, STROKE STATE INFORMATION PROVIDING DEVICE, AND STROKE STATE INFORMATION PROVIDING PROGRAM

(71) Applicants: Ohara Pharmaceutical Co., Ltd., Koka-shi Shiga (JP); Parkinson Laboratories Co., Ltd., Tokyo (JP); Juntendo Educational Foundation, Tokyo (JP)

(72) Inventors: Nobutaka Hattori, Tokyo (JP); Kazuo Yamashiro, Tokyo (JP); Yuji Ueno, Tokyo (JP); Nobukazu Miyamoto, Tokyo (JP); Mika Asari, Tokyo (JP); Kazuto Ono, Koka-shi Shiga (JP); Takashi Someda, Kyoto-shi, Kyoto (JP); Naoki Kitora, Kyoto-shi, Kyoto (JP); Yoshihiko Iwao, Kyoto-shi, Kyoto (JP); Keiichiro Takahashi, Kyoto-shi, Kyoto (JP)

(21) Appl. No.: 18/022,716

(22) PCT Filed: Sep. 9, 2021
(86) PCT No.: PCT/JP2021/033112

§ 371 Date: Jan. 2, 2024

(30) Foreign Application Priority Data
Sep. 11, 2020 (JP) 2020-152645

(51) Int. Cl. G16H 50/20; A61B 5/00; A61B 5/02; G06T 7/00; G06T 7/73

(52) U.S. Cl. CPC G16H 50/20; A61B 5/02007; A61B 5/4064; G06T 7/0012; G06T 7/73; A61B 5/742; G06T 2207/10081; G06T 2207/20081; G06T 2207/30016; G06T 2207/30101; G06T 2207/30242

(57) ABSTRACT

[Problems]

Provision of a system for assisting diagnosis/treatment of stroke which can easily provide supportive information so that the type of stroke can be determined appropriately and corresponding treatment strategy can be implemented.

[Solutions]

When the device for assisting diagnosis/treatment of stroke 101 acquires information of CT images, DWI, ADCmap, and MRA images indicating state of stroke from the device for providing information about state of stroke 103, it uses the acquired information of images and the information about laboratory findings to determine the type of stroke in a unit of stroke type determiner trained to use the predetermined information of images and the predetermined information about laboratory findings to determine the type of stroke corresponding thereto. The device for providing information about state of stroke 103 provides each information of images indicating state of stroke of a subject being diagnosed, to the device for assisting diagnosis/treatment of stroke 101, acquires and displays the type of stroke and therapeutic strategy determined from the device for assisting diagnosis/treatment of stroke 101.

### TECHNICAL FIELD

The present invention relates to a system for assisting diagnosis/treatment of stroke, specifically a system which can easily provide supportive information so that the type of stroke can be determined appropriately and corresponding treatment strategy can be implemented.

### BACKGROUND

A conventional watch over system is described using a device of medical image processor P100 shown in FIG. 23. The device of medical image processor P100 is characterized by comprising a data input unit 21 to which blood flow in a local brain tissue, which is local blood flow in a brain tissue of a subject, calculated based on medical images obtained by photographing the brain of a subject, is input, and a unit of cerebral infarction index calculator 32 which calculates cerebral infarction indices obtained by digitizing the recoverability of the subject's brain tissue over time based on the calculated blood flow in the local brain tissue.

Thus, information for determining a necessary treatment to cerebral infarction is provided by visualizing the progress of cerebral infarction at each site of the brain, (see patent literature 1).

### PRIOR ART LITERATURE

### Patent Literature

1. JP 2016-73542

### SUMMARY OF THE INVENTION

### Problems to be Solved by the Invention

The device of medical image processor P100 described above needs to be improved in some points as follows. The device of medical image processor P100 calculates the recoverability based on a local blood flow in a subject's brain tissue. Therefore, it is premised on application to recanalization therapy which removes thrombi formed in arteries of the brain and restores blood flow in brain tissue that is in ischemia.

On the other hand, there are several types of strokes that do not accompanied with intracerebral hemorrhage, including non-cardiogenic lacunar infarction, non-cardiogenic atherothrombotic cerebral infarction, and cardiogenic cerebral embolism, and therapeutic strategies are different depending on the type of cerebral infarction. Thus, it is difficult for a physician or the like who does not have sufficient knowledge about stroke to determine properly the type of stroke and implement corresponding therapeutic strategy.

Accordingly, it is an object of the present invention to provide a system for assisting diagnosis/treatment of stroke which can easily provide supportive information so that the type of stroke can be determined appropriately and corresponding treatment strategy can be implemented.

The object of the present invention is achieved by the following inventions.

(1) A system for assisting diagnosis/treatment of stroke which has a device for assisting diagnosis/treatment of stroke and a device for providing information about state of stroke, uses predetermined information of images that relates to stroke of a subject being diagnosed and information about laboratory findings that indicates the laboratory findings of the subject being diagnosed, to provide information for assisting diagnosis/treatment of stroke that assists diagnosis/treatment of stroke, and is characterized in that
the device for assisting diagnosis/treatment of stroke has
   a unit of image acquirer that acquires information of DWI, ADCmap, and MRA images as the information of images,
   a unit of laboratory finding acquirer that acquires the information about laboratory findings,
   a unit of stroke type determiner that has been trained to use predetermined information of images and predetermined information about laboratory findings to determine information about type of stroke corresponding thereto, and uses the acquired information of images and information about laboratory findings to determine the type of stroke,
   a unit of therapeutic strategy acquirer that acquires a corresponding therapeutic strategy from information about therapeutic strategy wherein the therapeutic strategy previously associated with the type of stroke is described, based on the determined type of stroke, and
   a unit of information provider for assisting diagnosis/treatment of stroke that provides the determined type of stroke and acquired therapeutic strategy as information for assisting diagnosis/treatment of stroke, and
the device for providing information about state of stroke has
   a unit of image provider that provides the information of images,
   a unit of information acquirer for assisting diagnosis/treatment of stroke that acquires the information for assisting diagnosis/treatment of stroke, and
   a display unit that displays the acquired information for assisting diagnosis/treatment of stroke.

(2) A device for assisting diagnosis/treatment of stroke which uses predetermined information of images that relates to stroke of a subject being diagnosed and information about laboratory findings that indicates the laboratory findings of the subject being diagnosed to assist diagnosis/treatment of stroke, and has
a unit of image acquirer that acquires information of DWI, ADCmap, and MRA images as the information of images,
a unit of laboratory finding acquirer that acquires the information about laboratory findings,
a unit of stroke type determiner that has been trained to use predetermined information of images and predetermined information about laboratory findings to determine information about type of stroke corresponding thereto, and uses the acquired information of images and information about laboratory findings to determine the type of stroke,
a unit of therapeutic strategy acquirer that acquires a corresponding therapeutic strategy from information about therapeutic strategy wherein the therapeutic strategy previously associated with the type of stroke is described, based on the determined type of stroke, and
a unit of information provider for assisting diagnosis/treatment of stroke that provides the determined type of stroke and acquired therapeutic strategy as information for assisting diagnosis/treatment of stroke.

(3) A device for assisting diagnosis/treatment of stroke characterized in that
furthermore, the unit of stroke type determiner in the device for assisting diagnosis/treatment of stroke according to (2) uses the information about infarction regions wherein the size, number, and position of the infarction region, responsible vessels for the infarction region, and their number are determined and the information about occlusions/stenoses wherein the presence or absence of intracranial stenoses is determined, for a cerebral infarction region where a cerebral infarction is occurring, by using the information of DWI, ADCmap, and MRA images, to determine the type of cerebral infarction, and
furthermore, the unit of stroke type determiner has been trained to determine from the predetermined information about infarction regions and the predetermined information about occlusions/stenoses, the type of cerebral infarction corresponding thereto.

(4) A device for assisting diagnosis/treatment of stroke characterized in that
furthermore, the unit of stroke type determiner in the device for assisting diagnosis/treatment of stroke according to (3) has a unit of cerebral infarction region determiner that uses the information of DWI and ADCmap to determine a cerebral infarction region where a cerebral infarction is occurring, and
the unit of cerebral infarction determiner has been trained to determine from the predetermined information of DWI and ADCmap, the cerebral infarction region corresponding thereto.

(5) A device for assisting diagnosis/treatment of stroke characterized in that
furthermore, the unit of stroke type determiner in the device for assisting diagnosis/treatment of stroke according to (4) has a unit of responsible vessel determiner that determines responsible vessels from the information about three-dimensional position of the predetermined cerebral infarction regions, and
the unit of responsible vessel determiner has been trained to be able to determine from the information about three-dimensional position of the predetermined cerebral infarction regions, the responsible vessels corresponding thereto.

(6) A program for assisting diagnosis/treatment of stroke that makes a computer function in the device for assisting diagnosis/treatment of stroke that uses predetermined information of images that relates to stroke of a subject being diagnosed and information about laboratory findings that indicates the laboratory findings of the subject being diagnosed, to assist diagnosis/treatment of stroke, as
a unit of image acquirer that acquires information of DWI, ADCmap, and MRA images as the information of images,
a unit of laboratory finding acquirer that acquires the information about laboratory findings,
a unit of stroke type determiner that has been trained to use predetermined information of images and predetermined information about laboratory findings to determine information about type of stroke corresponding thereto, and uses the acquired information of images and information about laboratory findings to determine the type of stroke,
a unit of therapeutic strategy acquirer that acquires a corresponding therapeutic strategy from information about therapeutic strategy wherein the therapeutic strategy previously associated with the type of stroke is described, based on the determined type of stroke, and
a unit of information provider for assisting diagnosis/treatment of stroke that provides the determined type of stroke and acquired therapeutic strategy as information for assisting diagnosis/treatment of stroke.

(7) A model for determination of the type of cerebral infarction, characterized by making a computer function as a unit of stroke type determiner that uses the information about infarction regions wherein the size, number, and position of the infarction region, responsible vessels for the infarction region, and their number are determined and the information about occlusions/stenoses wherein the presence or absence of intracranial stenoses is determined, for a cerebral infarction region where a cerebral infarction is occurring, by using the information of DWI, ADCmap, and MRA images, to determine the type of cerebral infarction, and that
furthermore, the unit of stroke type determiner has been trained to determine from the predetermined information about infarction regions and the predetermined information about occlusions/stenoses, the type of cerebral infarction corresponding thereto.

(8) A model for determination of cerebral infarction regions, characterized by making a computer function as a unit of image acquirer that acquires information of DWI and ADCmap of the brain and a unit of cerebral infarction region determiner that determines a cerebral infarction region where a cerebral infarction is occurring by using the information of DWI and ADCmap, and that
the unit of cerebral infarction determiner has been trained to determine from the predetermined information of DWI and ADCmap, a cerebral infarction region corresponding thereto.

(9) A model for determination of responsible vessels, characterized by making a computer function as a unit of responsible vessel determiner that determines responsible vessels from the information about three-dimensional position of a cerebral infarction region where a cerebral infarction is occurring, and that
the unit of responsible vessel determiner has been trained to be able to determine from the predetermined information about three-dimensional position of the cerebral infarction regions, the responsible vessels corresponding thereto.

### Effects of the Invention

The means for solving the problems in the present invention and effects of the invention are shown below.

The system for assisting diagnosis/treatment of stroke according to present invention has a device for assisting diagnosis/treatment of stroke and a device for providing information about state of stroke, and uses predetermined information of images that relates to stroke of a subject being diagnosed and information about laboratory findings that indicates the laboratory findings of the subject being diagnosed, to provide information for assisting diagnosis/treatment of stroke that assists diagnosis/treatment of stroke. The device for assisting diagnosis/treatment of stroke has a unit of image acquirer that acquires information of DWI, ADCmap, and MRA images as the information of images, a unit of laboratory finding acquirer that acquires the information about laboratory findings, a unit of stroke type determiner that has been trained to use predetermined information of images and predetermined information about laboratory findings to determine information about type of stroke corresponding thereto, and uses the acquired information of images and information about laboratory findings to determine the type of stroke, a unit of therapeutic strategy acquirer that acquires a corresponding therapeutic strategy from information about therapeutic strategy wherein the therapeutic strategy previously associated with the type of stroke is described, based on the determined type of stroke, and a unit of information provider for assisting diagnosis/treatment of stroke that provides the determined type of stroke and acquired therapeutic strategy as information for assisting diagnosis/treatment of stroke. The device for providing information about state of stroke has a unit of image provider that provides the information of images, a unit of information acquirer for assisting diagnosis/treatment of stroke that acquires the information for assisting diagnosis/treatment of stroke, and a display unit that displays the acquired information for assisting diagnosis/treatment of stroke.

As a result, information necessary for treatment can be easily provided to those who do not have sufficient knowledge about treatment of stroke by using predetermined information of images and information about laboratory findings.

The device for assisting diagnosis/treatment of stroke according to present invention uses predetermined information of images that relates to stroke of a subject being diagnosed and information about laboratory findings that indicates the laboratory findings of the subject being diagnosed to assist diagnosis/treatment of stroke, and has a unit of image acquirer that acquires information of DWI, ADCmap, and MRA images as the information of images, a unit of laboratory finding acquirer that acquires the information about laboratory findings, a unit of stroke type determiner that has been trained to use predetermined information of images and predetermined information about laboratory findings to determine information about type of stroke corresponding thereto, and uses the acquired information of images and information about laboratory findings to determine the type of stroke, a unit of therapeutic strategy acquirer that acquires a corresponding therapeutic strategy from information about therapeutic strategy wherein the therapeutic strategy previously associated with the type of stroke is described, based on the determined type of stroke, and a unit of information provider for assisting diagnosis/treatment of stroke that provides the determined type of stroke and acquired therapeutic strategy as information for assisting diagnosis/treatment of stroke.

As a result, the type of stroke that can be estimated from each information of images can be obtained by simply providing information of images and information about laboratory findings. That is, even those who do not have expert knowledge about stroke can be assisted so as to be able to diagnose appropriately the type of stroke and perform its treatment.

In the device for assisting diagnosis/treatment of stroke according to present invention, the unit of stroke type determiner is characterized by further using the information about infarction regions wherein the size, number, and position of the infarction region, responsible vessels for the infarction region, and their number are determined and the information about occlusions/stenoses wherein the presence or absence of intracranial stenoses is determined, for a cerebral infarction region where a cerebral infarction is occurring, by using the information of DWI, ADCmap, and MRA images, to determine the type of cerebral infarction, and furthermore, having been trained to determine from the predetermined information about infarction regions and the predetermined information about occlusions/stenoses, the type of cerebral infarction corresponding thereto.

As a result, the type of stroke can be determined more accurately, since a unit of stroke type determiner trained by using existing information about infarction regions and occlusions/stenoses is used.

In the device for assisting diagnosis/treatment of stroke according to present invention, the unit of stroke type determiner is characterized by having a unit of cerebral infarction region determiner that uses the information of DWI and ADCmap to determine a cerebral infarction region where a cerebral infarction is occurring by, and that the unit of cerebral infarction determiner has been trained to determine from the predetermined information of DWI and ADCmap, the cerebral infarction region corresponding thereto.

As a result, cerebral infarction regions can be determined more easily and accurately, since a unit of cerebral infarction region determiner trained by using existing information of DWI and ADCmap is used.

In the device for assisting diagnosis/treatment of stroke according to present invention, the unit of stroke type determiner is characterized by having a unit of responsible vessel determiner that determines responsible vessels from the information about three-dimensional position of the predetermined cerebral infarction regions, and that the unit of responsible vessel determiner has been trained to be able to determine from the information about three-dimensional position of the predetermined cerebral infarction regions, the responsible vessels corresponding thereto.

As a result, the responsible vessels corresponding to the information about three-dimensional position of cerebral infarction regions can be determined more easily and accurately, since a unit of responsible vessel determiner trained by using the existing information about three-dimensional position of cerebral infarction regions is used.

The program for assisting diagnosis/treatment of stroke according to present invention makes a computer function in the device for assisting diagnosis/treatment of stroke that uses predetermined information of images that relates to stroke of a subject being diagnosed and information about laboratory findings that indicates the laboratory findings of the subject being diagnosed, to assist diagnosis/treatment of stroke, as a unit of image acquirer that acquires information of DWI, ADCmap, and MRA images as the information of images, a unit of laboratory finding acquirer that acquires the information about laboratory findings, a unit of stroke type determiner that has been trained to use predetermined information of images and predetermined information about laboratory findings to determine information about type of stroke corresponding thereto, and uses the acquired information of images and information about laboratory findings to determine the type of stroke, a unit of therapeutic strategy acquirer that acquires a corresponding therapeutic strategy from information about therapeutic strategy wherein the therapeutic strategy previously associated with the type of stroke is described, based on the determined type of stroke, and a unit of information provider for assisting diagnosis/treatment of stroke that provides the determined type of stroke and acquired therapeutic strategy as information for assisting diagnosis/treatment of stroke.

As a result, the state of stroke that can be estimated from each information of images can be obtained by simply providing predetermined information of images and information about laboratory findings to a computer. That is, even those who do not have expert knowledge about stroke can be assisted so as to be able to determine the state of stroke easily.

The model for determination of the type of cerebral infarction of the present invention is characterized by making a computer function as a unit of stroke type determiner that uses the information about infarction regions wherein the size, number, and position of the infarction region, responsible vessels for the infarction region, and their number are determined and the information about occlusions/stenoses wherein the presence or absence of intracranial stenoses is determined, for a cerebral infarction region where a cerebral infarction is occurring, by using the information of DWI, ADCmap, and MRA images, to determine the type of cerebral infarction, and that furthermore, the unit of stroke type determiner has been trained to determine from the predetermined information about infarction regions and the predetermined information about occlusions/stenoses, the type of cerebral infarction corresponding thereto.

As a result, a model for determination of the type of stroke trained by using existing information about infarction regions and occlusions/stenoses can be constructed in a computer, and the type of stroke can be determined more accurately by using the constructed model for determination of the type of stroke.

The model for determination of cerebral infarction regions according to present invention is characterized by making a computer function as a unit of image acquirer that acquires information of DWI and ADCmap of the brain and a unit of cerebral infarction region determiner that determines a cerebral infarction region where a cerebral infarction is occurring by using the information of DWI and ADCmap, and that the unit of cerebral infarction determiner has been trained to determine from the predetermined information of DWI and ADCmap, a cerebral infarction region corresponding thereto.

As a result, a model for determination of cerebral infarction regions trained by using existing information of DWI and ADCmap can be constructed in a computer, and cerebral infarction regions can be determined more easily and accurately by using the constructed model for determination of cerebral infarction regions.

The model for determination of responsible vessels according to present invention is characterized by making a computer function as a unit of responsible vessel determiner that determines responsible vessels from the information about three-dimensional position of a cerebral infarction region where a cerebral infarction is occurring, and that the unit of responsible vessel determiner has been trained to be able to determine from the information about three-dimensional position of the predetermined cerebral infarction regions, the responsible vessels corresponding thereto.

As a result, a model for determination of responsible vessels trained by using existing information about three-dimensional position of a cerebral infarction region can be constructed in a computer, and the responsible vessels corresponding to the information about three-dimensional position of a cerebral infarction region can be determined more easily and accurately by using the constructed model for determination of responsible vessels.

### BRIEF DESCRIPTION OF THE DRAWINGS

FIG. 1 shows a system for assisting diagnosis/treatment of stroke 100, which is an example of the system for assisting diagnosis/treatment of stroke according to the present invention.

FIG. 2 shows the configuration of hardware of the device for assisting diagnosis/treatment of stroke 101.

FIG. 3 shows the configuration of hardware of the device for providing information about state of stroke 103.

FIG. 4 shows the data structure of information about state of stroke.

FIG. 5 shows the data structure of information about conditions of a subject being diagnosed.

FIG. 6A-D shows an example of information of a CT image (A), a DWI (B), an ADCmap (C), and an MRA image (D).

FIG. 7 shows the data structure of information about laboratory findings.

FIG. 8 shows the data structure of information about therapeutic strategies.

FIG. 9 shows the data structure of information for assisting diagnosis/treatment of stroke.

FIG. 10 shows the data structure of information for determining type of stroke.

FIG. 11 is a flowchart showing the operation of the device for providing information about state of stroke 103.

FIG. 12 shows a screen for sending the information about state of stroke displayed by the device for providing information about state of stroke 103.

FIG. 13 is a flowchart showing the operation of the device for assisting diagnosis/treatment of stroke 101.

FIG. 14 is a flowchart showing the processing for determination of intracranial hemorrhage.

FIG. 15 is a flowchart showing the processing for generation of information about infarction regions.

FIG. 15a shows an example of information about infarction regions.

FIG. 16 is a flowchart showing the processing for extraction of infarction regions.

FIG. 17 is a flowchart showing the processing for determination of occlusions/stenoses.

FIG. 18 is a flowchart showing the processing for determination of occlusions.

FIG. 19 shows division of information of an MRA image during the processing for determination of occlusions.

FIG. 20 is a flowchart showing the processing for determination of stenoses.

FIG. 21 is a flowchart showing the processing for generation of information for assisting diagnosis/treatment of stroke.

FIG. 22 shows a screen for displaying information for assisting diagnosis/treatment of stroke displayed by the device for providing information about state of stroke 103.

FIG. 23 shows a conventional system for assisting diagnosis/treatment of cerebral infarction.

### DETAILED DESCRIPTION OF THE INVENTION

The example of the present invention will be described in detail as follows with reference to drawings.

### Example 1

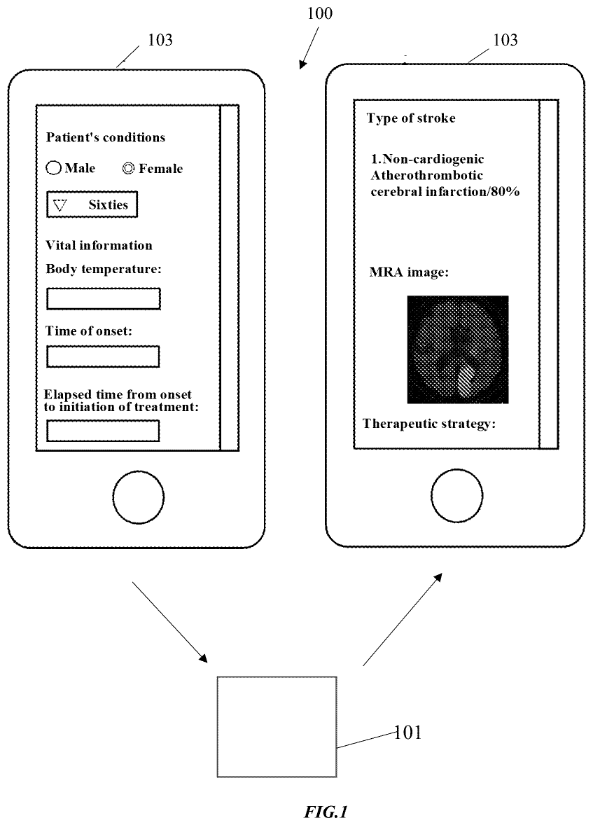

The device for assisting diagnosis/treatment of stroke according to the present invention is described by using the device for assisting diagnosis/treatment of stroke 100 shown in FIG. 1 as an example.

I. Configuration of Hardware

1. The System for Assisting Diagnosis/Treatment of Stroke 100

The system for assisting diagnosis/treatment of stroke 100 comprises a device for assisting diagnosis/treatment of stroke 101 and a device for providing information about state of stroke 103. The device for assisting diagnosis/treatment of stroke 101 and the device for providing information about state of stroke 103 are connected by a predetermined network, and can send and receive information to each other via the predetermined network.

When the device for assisting diagnosis/treatment of stroke 101 acquires from the device for providing information about state of stroke 103, the information of CT images (described later), DWI (described below), MRA images (described below) that indicate the state of stroke, information about conditions of a subject being diagnosed who is a patient, and information about laboratory findings based on various tests, it estimates the type of stroke based on accumulated information about diagnosis/treatment of stroke wherein the data of diagnosis/treatment of stroke obtained so far are accumulated from the acquired information of CT images, DWI, MRA images, information about conditions of the subject being diagnosed, and information about laboratory findings, and estimates corresponding therapeutic strategy.

The device for providing information about state of stroke 103 provides the information of CT images, DWI, ADCmap, MRA images, information about conditions of a subject being diagnosed, and information about laboratory findings that relate to the state of strike of the subject being diagnosed who is likely to be in a state of stroke, to the device for assisting diagnosis/treatment of stroke 101 via a network. In addition, the device for providing information about state of stroke 103 acquires from the device for assisting diagnosis/treatment of stroke 101, the type of stroke and the therapeutic strategy estimated from the sent information of CT images, DWI, ADCmap, MRA images, information about conditions of the subject being diagnosed, and information about laboratory findings and displays them.

As a result, users of the device for providing information about state of stroke 103 can easily obtain necessary information for treatment of stroke, even if they do not have sufficient knowledge about its treatment.

2. The Device for Assisting Diagnosis/Treatment of Stroke 101

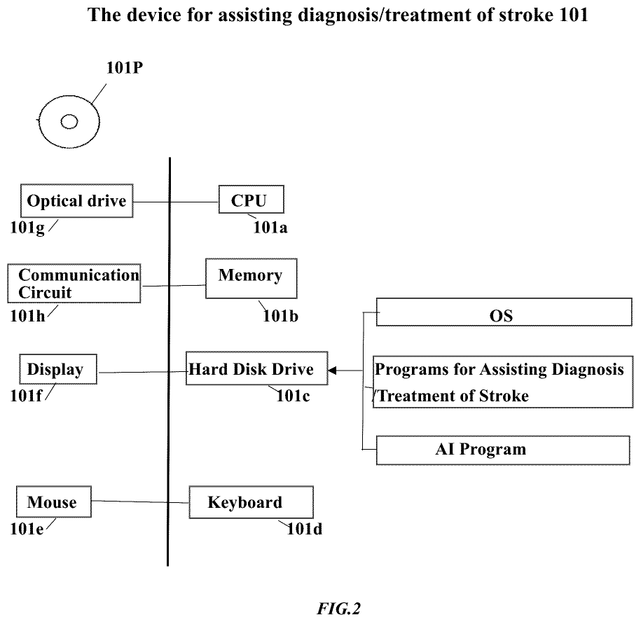

The configuration of hardware of the device for assisting diagnosis/treatment of stroke 101 is described with reference to FIG. 2. The device for assisting diagnosis/treatment of stroke 101 comprises a CPU 101a, memory 101b, a hard disk drive 101c (hereinafter referred to as HDD 101c), a keyboard 101d, a mouse 101e, a display 101f, an optical drive 101g, and a communication circuit 101h.

The CPU 101a performs processing based on an operating system (OS) recorded in the HDD 101c and other applications such as the program for assisting diagnosis/treatment of stroke. The memory 101b provides working regions for the CPU 101a. The HDD 101c records and retains an operating system (OS), and programs of other applications such as the program for assisting diagnosis/treatment of stroke and AI (artificial intelligence) programs, and various data. The AI programs are selected as appropriate according to the characteristics of each existing programs.

The keyboard 101d and the mouse 101e receive external commands. The display 101f displays images, such as user interface. The optical drive 101g reads data from optical media. For example, it reads the program for assisting diagnosis/treatment of stroke from the optical media 101p on which the program for assisting diagnosis/treatment of stroke is recorded. In addition, it reads programs of other applications from other optical media. The communication circuit 101h is connected to a predetermined network and sends/receives information to/from external communication devices such as the device for providing information about state of stroke 103.

3. The Device for Providing Information about State of Stroke 103

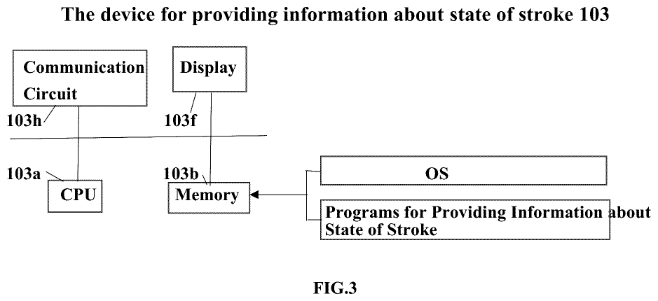

The configuration of hardware of the device for providing information about state of stroke 103 is described with reference to FIG. 3. The device for providing information about state of stroke 103 comprises a CPU 103a, a memory 103b, a display 103f, and a communication circuit 103h. It is noted that a smartphone can be used as the device for providing information about state of stroke 103.

The CPU 103a performs processing based on an operating system (OS) recorded in the memory 103b and other applications such as the program for providing information about state of stroke. The memory 103b provides a working area for the CPU 103a. The memory 103b records and retains an operating system (OS), programs of other applications such as the program for providing information about state of stroke, and various data.

The display 103f displays images such as user interface. The communication circuit 101h is connected to a predetermined network and sends/receives information to/from external communication devices such as the device for assisting diagnosis/treatment of stroke 101.

II. Information Used

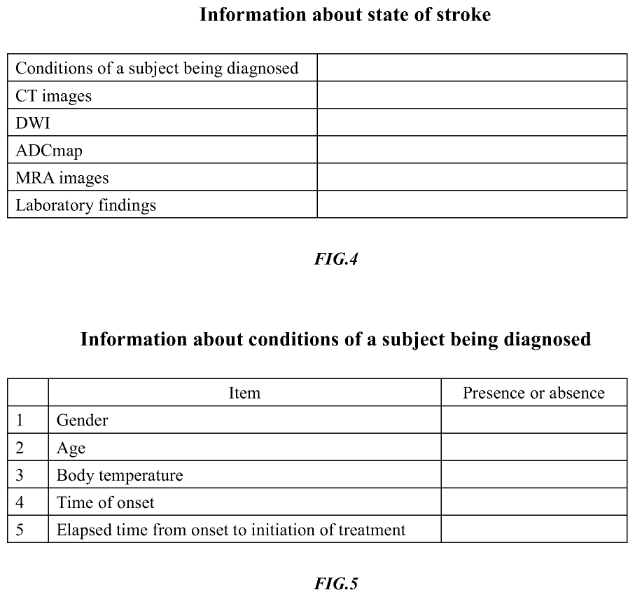

Information used in the watch over system 100 is described with reference to FIGS. 4 to 11

1. Information about State of Stroke

The information about state of stroke indicates the state of stroke of a subject being diagnosed, that is, a patient. The data structure of the information about state of stroke is shown in FIG. 4. The information about state of stroke has the regions describing conditions of a subject being diagnosed, CT images, a DWI images, ADCmap, MRA images, and laboratory findings.

In the region describing conditions of a subject being diagnosed, the information about conditions of a subject being diagnosed that indicates conditions of the subject being diagnosed is described. The gender, age, body temperature of the subject being diagnosed, time of onset, elapsed time from the onset to initiation of treatment, and the like are described in the region describing conditions of a subject being diagnosed.

In the region describing CT images, information of CT images of a subject being diagnosed is described. In the region describing DWI images, information of DWI of a subject being diagnosed is described. In the region describing ADCmap, information of ADCmap of a subject being diagnosed is described. In the region describing MRA images, information of MRA images of a subject being diagnosed is described.

In the region describing laboratory findings, information about laboratory findings related to predetermined items determined from examinations, etc. performed on a subject being diagnosed is described. The information described in each description region is described below.

(1) Information about Conditions of a Subject being Diagnosed

The information about conditions of a subject being diagnosed relates to examinations determined from those performed on a subject being diagnosed whose conditions are to be determined, by using the system for assisting diagnosis/treatment of stroke 100.

The data structure of the information about conditions of a subject being diagnosed is shown in FIG. 5. The information about conditions of a subject being diagnosed has a region describing the presence or absence of atrial fibrillation, transient atrial fibrillation, use of prosthetic valve, left ventricular thrombus, and sinus dysfunction syndrome.

(2) Information of CT Images

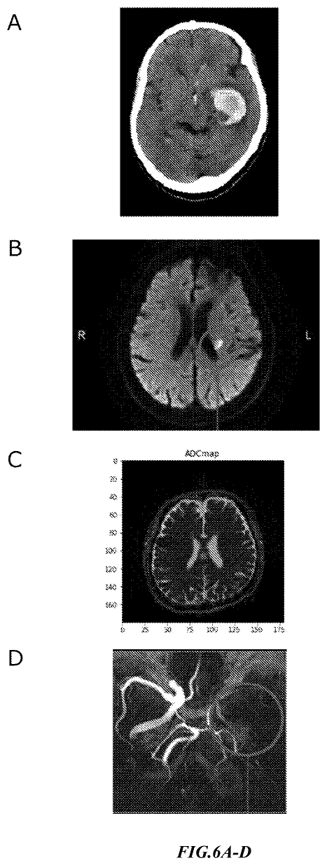

The information of CT images indicates brain images of a subject being diagnosed acquired by using CT (computer tomography). The information of CT images is generated with a CT device operated by a physician or the like. An example of information of CT images is shown in FIG. 6A.

(3) Information of DWI

The information of DWI indicates a type of sequence of nuclear magnetic resonance imaging wherein the diffusion motion of water molecules is imaged. The DWI (diffusion weighted image) is generated with a DWI device operated by a physician or the like. An example of information of DWI is shown in FIG. 6B.

(4) Information of ADCmap

The information of ADCmap shows imaged apparent diffusion coefficient (ADC) obtained from a plurality of DWI obtained by adding a dephasing effect by changing the strength and application time of the gradient magnetic field in the pulse sequence of the spin echo method. The information of ADCmap is generated with an ADCmap device operated by a physician or the like. An example of information of ADCmap is shown in FIG. 6C.

(5) Information of MRA Images

The information of MRA images shows vessel images of a subject being diagnosed acquired by using MRA (magnetic resonance angiography). The information of MRA images is generated with an MRA device operated by a physician or the like. An example of information of MRA images is shown in FIG. 6D.

(6) Information about Laboratory Findings

The information about laboratory findings relates to state of a disease determined from examinations performed on a subject being diagnosed, that is, a patient, whose conditions are to be determined, by using the system for assisting diagnosis/treatment of stroke 100.

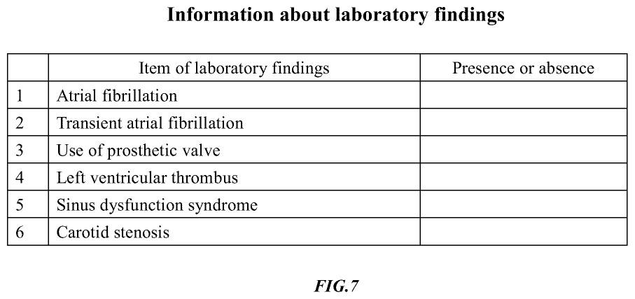

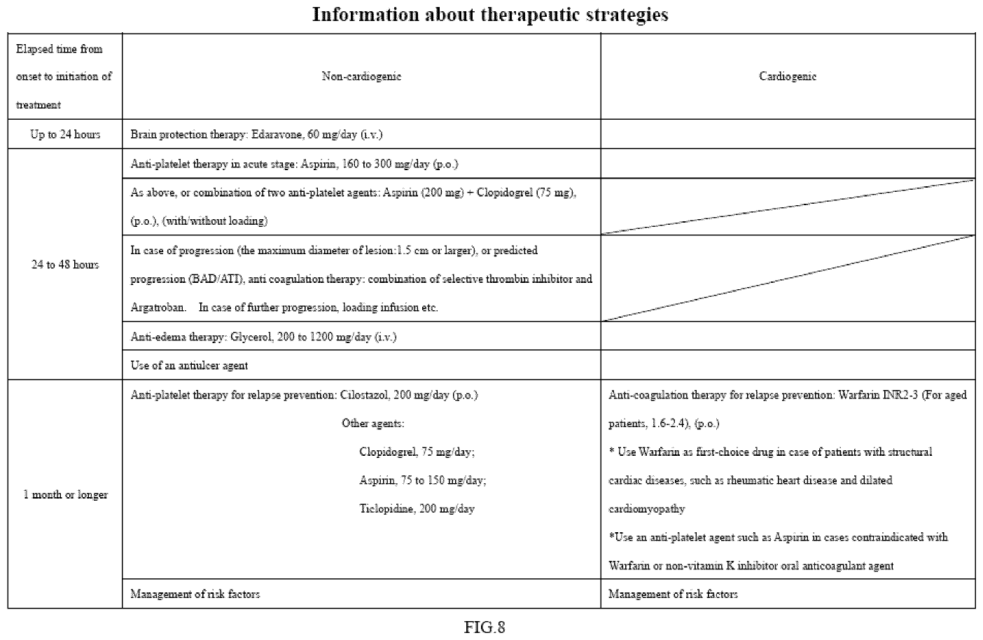

The data structure of the information about laboratory findings is shown in FIG. 7. The information about laboratory findings has a region describing the presence or absence of atrial fibrillation, transient atrial fibrillation, use of prosthetic valve, left ventricular thrombus, sinus dysfunction syndrome, and carotid stenosis. It is noted that the information about laboratory findings is acquired from medical interviews with a subject being diagnosed, various examinations, systemic findings, and the like.

2. Information about Therapeutic Strategies

The information about therapeutic strategies is a database wherein state of stroke and therapeutic strategies to be used for treatment are associated.

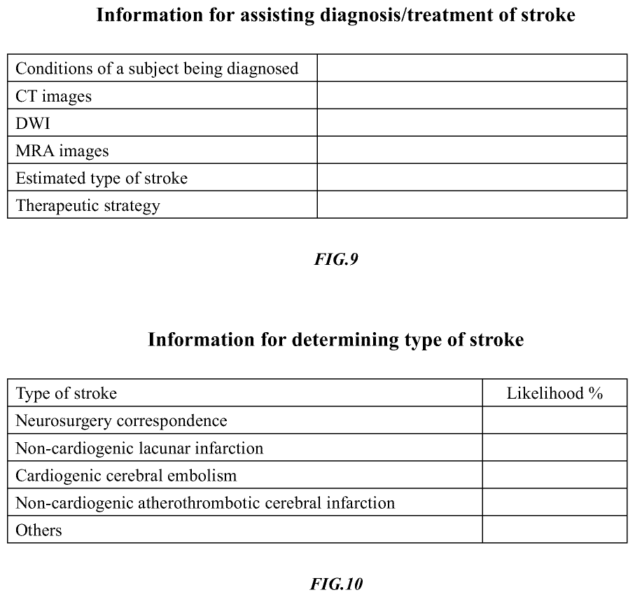

The data structure of the information about therapeutic strategies is shown in FIG. 8. The information about therapeutic strategies is classified based on state of stroke and elapsed time from the onset to initiation of treatment and described. For example, if the state of stroke is “non-cardiac” and it is within 24 hours after onset, “non-cardiac” is described in a horizontal column and “within 24H” is described in a longitudinal column as therapeutic strategy to be performed.

3. Information for Assisting Diagnosis/Treatment of Stroke

The information for assisting diagnosis/treatment of stroke indicates the type of stroke of a subject being diagnosed estimated from the information about state of stroke acquired from the subject being diagnosed and therapeutic strategy corresponding thereto. By providing the information for assisting diagnosis/treatment of stroke, a physician who is less inexperienced in the diagnosis and treatment of stroke is assisted so as to be able to perform appropriate diagnosis and treatment.

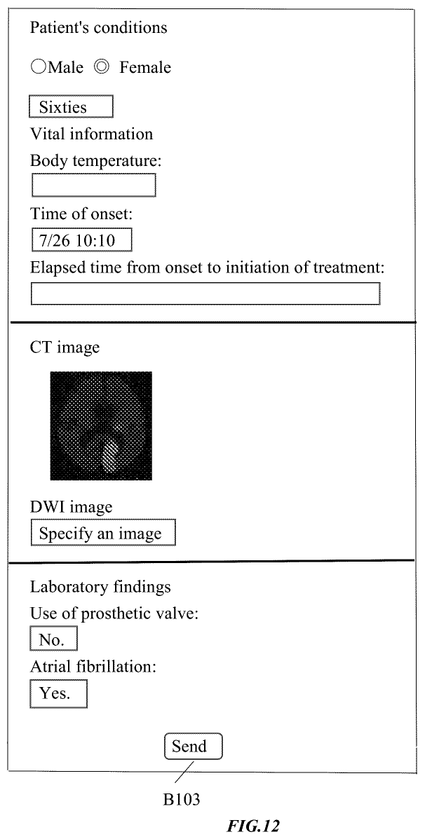

The data structure of the information for assisting diagnosis/treatment of stroke is shown in FIG. 10. The information for assisting diagnosis/treatment of stroke has the regions describing conditions of a subject being diagnosed, CT images, DWI, ADCmap, MRA images, estimated type of stroke, estimated laboratory findings of stroke, additional examinations, and therapeutic strategy. In the regions describing conditions of a subject being diagnosed, CT images, DWI, ADCmap, and MRA images, the information described in the regions describing conditions the subject being diagnosed, CT images, DWI, ADCmap, and MRA images of the information about state of stroke (see FIG. 4) is described.

In the region describing estimated type of stroke, information about determined type of stroke indicating the estimated type of stroke of a subject being diagnosed based on the information described in the regions describing conditions a subject being diagnosed, CT images, DWI, ADCmap, and MRA images of the information about state of stroke (see FIG. 4). The information about determined type of stroke will be described later.

In the information about therapeutic strategies, a therapeutic strategy extracted from the information about therapeutic strategies (see FIG. 8) corresponding to the estimated type of stroke of a subject being diagnosed is described.

(1) Information for Determining Type of Stroke

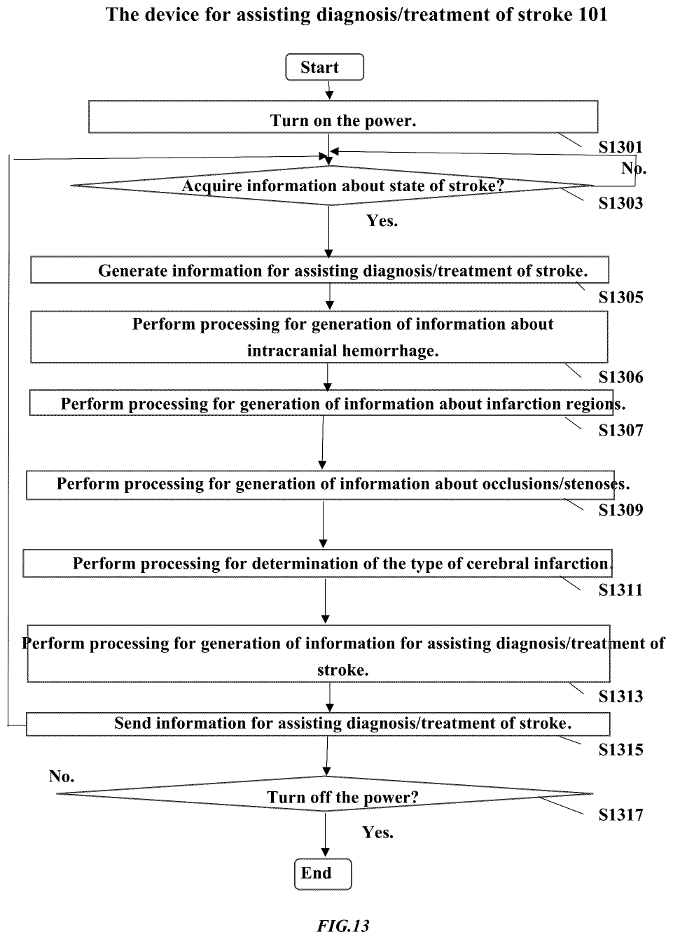

The data structure of the information for determining type of stroke is shown in FIG. 11. The information for determining type of stroke has the regions describing an estimated type of stroke and likelihood. In the regions describing an estimated type of stroke, a type of stroke estimated for a subject being diagnosed is described. As the estimated type of stroke, items of “neurosurgery correspondence”, “non-cardiogenic lacunar infarction”, “non-cardiogenic atherothrombotic cerebral infarction”, “cardiogenic cerebral embolism”, and “the other” are included. In the region describing likelihood, the likelihood (%) for each item in the region describing estimated type of stroke is described.

The “non-cardiogenic lacunar infarction” refers to the infarction not caused by a heart disease, but caused by thrombi generated in artery and clogging of fine arteries in the brain. In addition, the “non-cardiogenic atherothrombotic cerebral infarction (ATI)” refers to the infarction caused by the curing of carotid artery from the neck to the brain and relatively thick arteries in the brain (atherosclerosis). Furthermore, the “cardiogenic cerebral embolism (CE)” refers to the infarction caused by that the thrombi generated in the heart flow into the arteries in the brain to cause the vessels in the brain to be clogged.

II. Operation of the System for Assisting Diagnosis/Treatment of Stroke 100

The operation of the system for assisting diagnosis/treatment of stroke 100 is described with reference to FIGS. 11 to 19. A user such as a physician who utilizes the system for assisting diagnosis/treatment of stroke 100 uses the device for providing information about state of stroke 103 to send to the device for assisting diagnosis/treatment of stroke 101, the gender, age, body temperature of a subject being diagnosed who is a patient, time of onset, elapsed time from the onset to initiation of treatment, etc. as information about conditions of a subject being diagnosed, CT images of the subject being diagnosed as information of CT images, DWI images as information of DWI images, MRA images as information of MRA images, findings based on medical interviews with a subject being diagnosed and various examinations, and systemic findings as information about laboratory findings. The device for assisting diagnosis/treatment of stroke 101 estimates the state of stroke of the patient and the corresponding therapeutic strategy from the various information acquired and provides the information for assisting diagnosis/treatment of stroke for a physician to determine state of stroke and perform appropriate treatment.

1. Operation (1) of the Device for Providing Information about State of Stroke 103

The operation of the device for providing information about state of stroke 103 is described with reference to the flowchart shown in FIG. 11. Before utilizing the system for assisting diagnosis/treatment of stroke 100, a physician or the like keeps track of the patient's conditions. Besides, the physician acquires CT images, DWI images, and MRA images of the patient with predetermined devices. Furthermore, the physician performs necessary examinations to obtain laboratory findings of the patient.

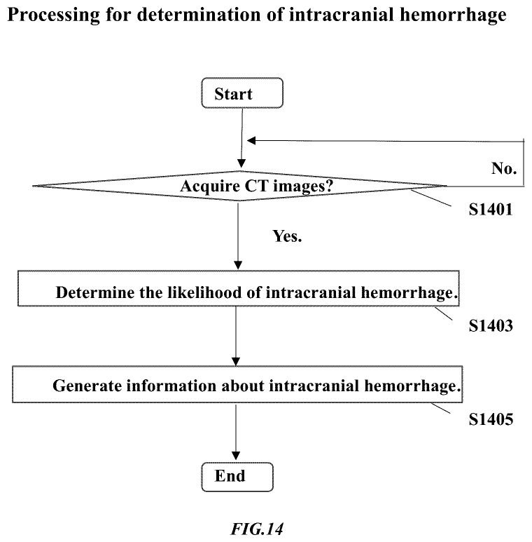

A user of the device for providing information about state of stroke 103 (referred to as a user hereinafter) activates the program for providing information about state of stroke of the device for providing information about state of stroke 103. When the CPU 103a of the device for providing information about state of stroke 103 determines that the program for providing information about state of stroke has been activated (S801), the CPU 103a displays an input screen (see FIG. 12) for inputting conditions of a patient who is a subject being diagnosed, the CT images, DWI images, and MRA images of the patient, and the laboratory findings of the patient (S803). The information about a patient's conditions includes for example, age, gender, and the like. An end user inputs each item according to the input screen and selects the sending button B103.

When the CPU 103a determines that the sending button B103 has been selected (S805), the CPU 103a sends the input conditions of a subject being diagnosed, the CT images, DWI images, and MRA images of the patient, and laboratory findings as information about state of stroke (see FIG. 4) to the device for assisting diagnosis/treatment of stroke 101 (S807). 2. Operation of the device for assisting diagnosis/treatment of stroke 101

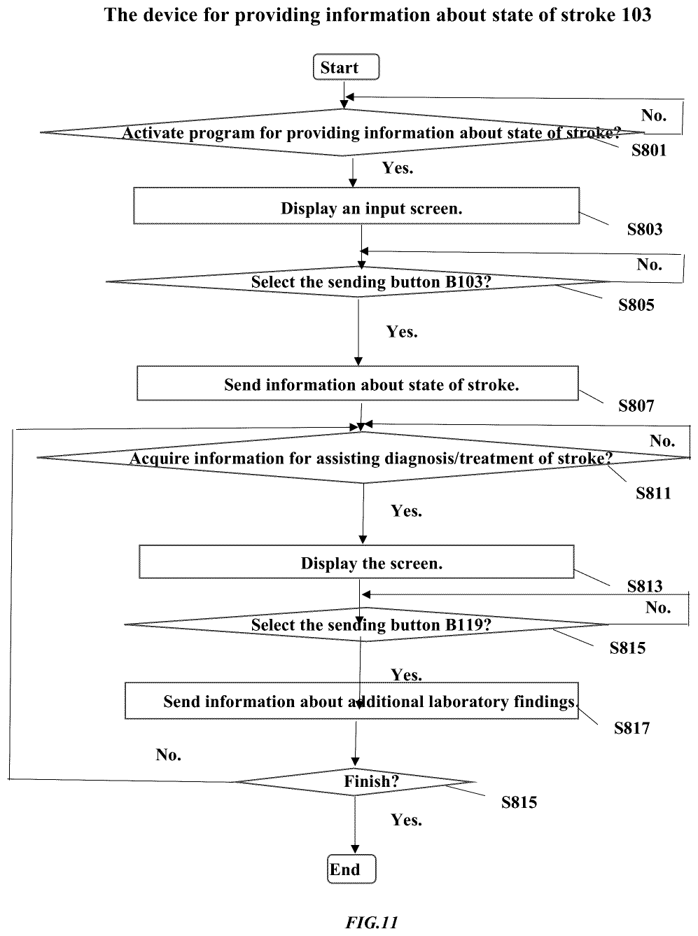

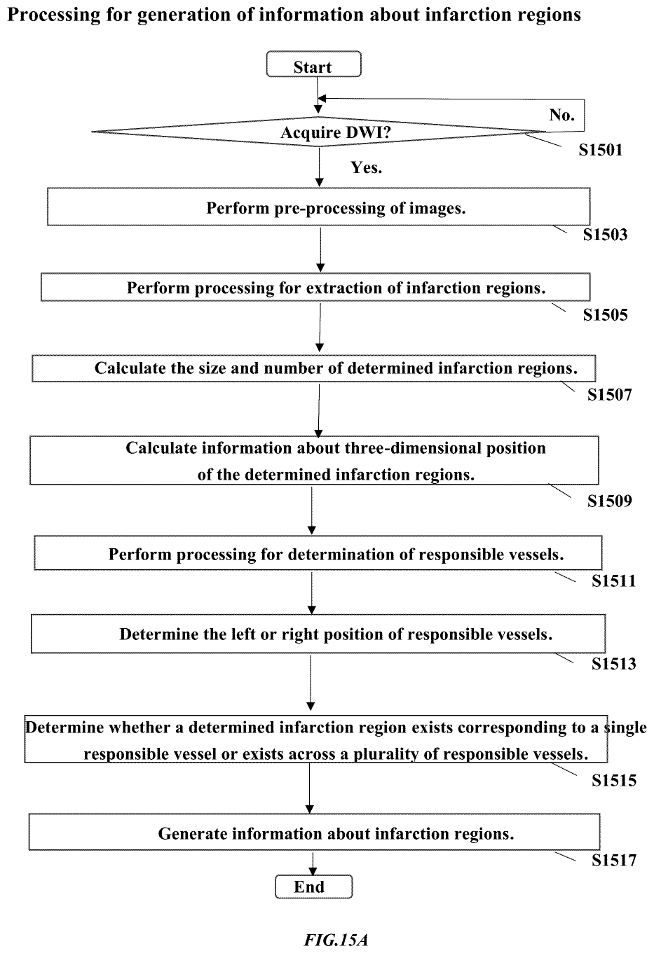

An overview of the operation of the device for assisting diagnosis/treatment of stroke 101 is described with reference to the flowchart shown in FIG. 13. When the CPU 101a of the device for assisting diagnosis/treatment of stroke 101 acquires the information about state of stroke from the device for providing information about state of stroke 103 (S1303) after the power is turned on (S1301), it generates the information for assisting diagnosis/treatment of stroke (see FIG. 9) corresponding to the acquired information about state of stroke (S1305). It is noted that at this stage, nothing is described in each region describing the information for assisting diagnosis/treatment of stroke.

The CPU 101a performs the processing for generation of information about intracranial hemorrhage based on the CT images acquired from the information about state of stroke to generate information about intracranial hemorrhage (S1306). In addition, the CPU 101a performs the processing for generation of information about infarction regions based on the DWI and ADCmap acquired from the information about state of stroke to generate information about infarction regions (S1307). Furthermore, the CPU 101a performs the processing for generation of information about occlusions/stenoses based on MRA to generate information about occlusions/stenoses (S1309). The CPU 101a performs the processing for determination of types of cerebral infarction based on the generated information about infarction regions, the information about occlusions/stenoses, and the information about the patient to generate information about the type of cerebral infarction (S1311).

The CPU 101a uses the information about the type of cerebral infarction to perform the processing for generation of information for assisting diagnosis/treatment of stroke and generate information for assisting diagnosis/treatment of stroke (S1313). The CPU 101a sends the generated information for assisting diagnosis/treatment of stroke to the device for providing information about state of stroke 103 (S1315).

The CPU 101a repeats the processing of steps S1303 to S1315 until the power is turned off.

The processing for generation of information about intracranial hemorrhage, the processing for generation of information about infarction regions, the processing for generation of information about occlusions/stenoses, the processing for determination of types of cerebral infarction, and the processing for generation of information for assisting diagnosis/treatment of stroke are described below.

1. The Processing for Generation of Information about Intracranial Hemorrhage

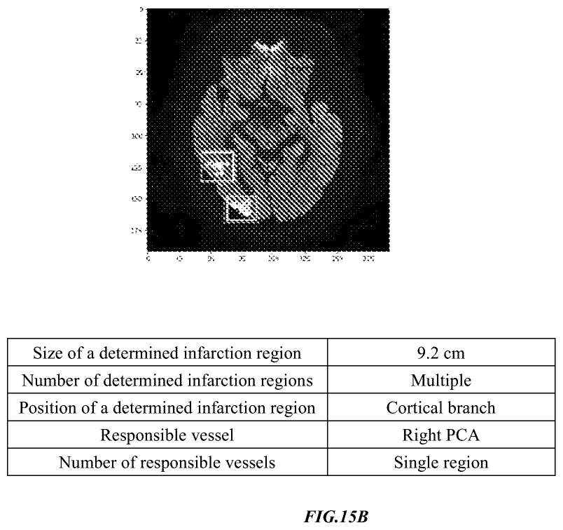

The processing for generation of information about intracranial hemorrhage is described with reference to the flowchart shown in FIG. 14. When the CPU 101a acquires information of CT images from the acquired information about state of stroke (S1401), it uses AI programs to determine the likelihood of the presence or absence of intracranial hemorrhage in the acquired information of CT images (S1403). Upon determining the presence or absence of intracranial hemorrhage, a model for determination of intracranial hemorrhage trained to calculate the likelihood of “intracranial hemorrhage” and “no intracranial hemorrhage” from the CT images has been constructed in the AI programs.

When the CPU 101a has calculated the likelihood of intracranial hemorrhage in step S1403, it generates information about intracranial hemorrhage wherein “intracranial bleeding” and “no intracranial bleeding” are paired with their likelihood, respectively (1405).

2. The Processing for Generation of Information about Infarction Regions

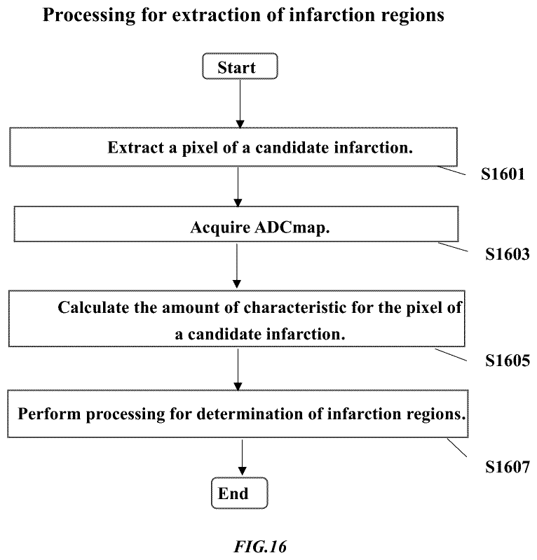

The processing for generation of information about infarction regions is described with reference to the flowchart shown in FIG. 15. The CPU 101a acquires DWIs from acquired supportive information (S1501). The CPU 101a performs pre-processing of images for correcting variation between images on the acquired DWIs (S1503).

Variation in DWI occurs due to differences in DWI devices such as manufacturers and models, individual differences such as thickness of vessel of subjects being imaged, conditions of subjects being imaged at the time of imaging, and the like. For this reason, in the pre-processing of images, image correction regarding brightness, rotation, and position, etc. on the acquired DWIs is performed to reduce the variation between the DWIs.

The CPU101a then performs a processing for extraction of infarction regions on the pre-processed DWIs (S1505).

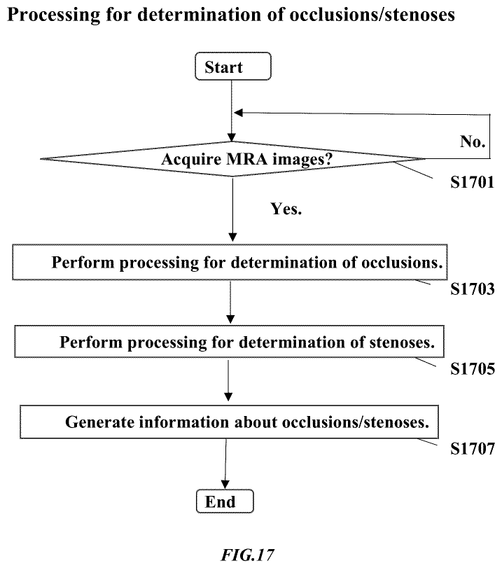

The processing for extraction of infarction regions is described with reference to FIG. 16. The processing for extraction of infarction regions is a processing of identifying and extracting the regions where cerebral infarction is occurring from DWIs. The CPU 101a extracts a pixel having a higher signal than the surroundings from the pre-processed DWIs as a pixel of candidate infarction (S1601). The CPU 101a acquires ADCmap (S1603). The CPU 101a calculates the amount of characteristic for the extracted pixel of candidate infarction (S1605).

As the amount of characteristic for the pixels of candidate infarction, information about three-dimensional position and left-right symmetry of the pixels of candidate infarction, pixel values of ADCmap pixels corresponding to pixels of candidate infarction of DWI (referred to as corresponding ADCmap candidate pixels hereinafter), and surrounding pixel values of the corresponding ADCmap candidate pixels are used.

DWI is an image of the degree of molecules' Brownian motion and is characterized in that the pixel value turns higher in an infarction region where diffusion is limited. On the other hand, since the DWI is a kind of T2-weighted image, the region with a high pixel value in a T2-weighted image may result in a higher pixel value even if the corresponding region of the DWI does not have any diffusion limit, which is referred to as T2 shine-through. Upon interpreting, in order to distinguish between the region where the infarction is actually occurring and the region of the T2 shine-through, a physician not only considers the DWIs but also ADCmap images at the same time. In the region where the infarction is occurring, the pixel value in DWI turns higher than the surroundings, while the pixel value in ADCmap turns lower than the surroundings. On the other hand, in the region of T2 shine-through, even if the pixel value in DWI is higher than the surroundings, the pixel value in ADCmap dose not turn lower than the surroundings. Therefore, the pixel values of not only DWI but also ADCmap are used as the amount of characteristic in order to distinguish the infarction region from the region of T2 shine-through.

The CPU 101a uses the calculated amount of characteristic to performs a processing for determination of infarction regions (S1607). The processing for determination of infarction regions is a processing of determining whether the regions of pixels of candidate infarction extracted from DWI are infarction regions. Gradient boosting decision trees (referred to as GBDT hereafter) are used as algorithm in the processing for determination of infarction regions.

It is noted that in the processing for determination of infarction regions, a model for determination of infarction regions trained to calculate infarction regions has been constructed in advance from DWI, ADCmap, and the amount of characteristic of several cases reviewed by physicians.

Returning to FIG. 15, the CPU 101a determines the size and number of infarction regions from the region determined to be the infarction regions in DWI (referred to as determined infarction regions hereafter) (S1507).

In addition, the CPU 101a calculates the information about three-dimensional position of the determined infarction regions (S1509). The CPU 101a uses the calculated information about three-dimensional position of the determined infarction regions to perform the processing for determination of responsible vessels (S1511).

The processing for determination of responsible vessels is a processing for determining, from the calculated information about three-dimensional position of the determined infarction regions, the position of determined infarction regions whether a determined infarction region is present in the cortical branch or in a narrow branch, and for determining responsible vessels. The responsible vessels are classified into six types, including basilar artery (BA), anterior inferior cerebellar artery (AICA), posterior inferior cerebellar artery (PICA), middle cerebral artery (MCA), anterior cerebral artery (ACA), and posterior cerebral artery (PCA). The vessels except basilar artery (BA) exist symmetrically on left and right sides, however, the left ones and right ones are not distinguished in the processing for determination of responsible vessels. Besides, decision trees are used as algorithm in the processing for determination of responsible vessels.

It is noted that in the processing for determination of responsible vessels, a model for determination of responsible vessels trained to calculate whether a determined infarction region is present in the cortical branch or in a narrow branch from the information about three-dimensional position of infarction regions, and responsible vessels for infarction regions has been constructed in advance.

The CPU 101a uses the X coordinates of the calculated information about three-dimensional position of determined infarction regions to determine the left and right position of responsible vessels (S1513). It is noted that the determination of the left and right position of responsible vessels is performed for the vessels except basilar artery (BA) determined in step S1711.

The CPU 101a uses the responsible vessels determined in step S1511 and the left and right position of the responsible vessels determined in step S1513 to determine whether a determined infarction region exists corresponding to a single responsible vessel or exists across a plurality of responsible vessels (S1515).

The CPU 101a uses the size and number of a determined infarction region determined in step S1507, the position and the responsible vessels of a determined infarction region determined in step S1511, and the number of responsible vessels determined in step S1515 to generate information about infarction regions (S1517).

An example of the generated information about infarction regions is shown in FIG. 15a. FIG. 15aA shows a DWI with determined infarction regions indicated in squares. In addition, FIG. 15aB shows the size, number, and position of determined infarction regions, responsible vessels, and their number corresponding to the DWI shown in FIG. 15A.

3. The Processing for Generation of Information about Occlusions/Stenoses

(1) Summary

When a vascular occlusion occurs, a vessel beyond a region where a vascular occlusion is occurring disappears from an image captured using magnetic resonance angiography (referred to as MRA images hereafter). Therefore, it is difficult to directly determine from MRA images the region where a vascular occlusion is occurring, which is a problem. On the other hand, when a vascular stenosis occurs, the vessel where the stenosis is occurring exists in MRA images. For this reason, the region where a vascular stenosis is occurring can be determined directly from MRA images. Therefore, due to the characteristic difference between vascular occlusions and stenoses in MRA images, the occurrence of vascular occlusions and stenoses is determined respectively by processing for determination of occlusions and processing for determination of stenoses.

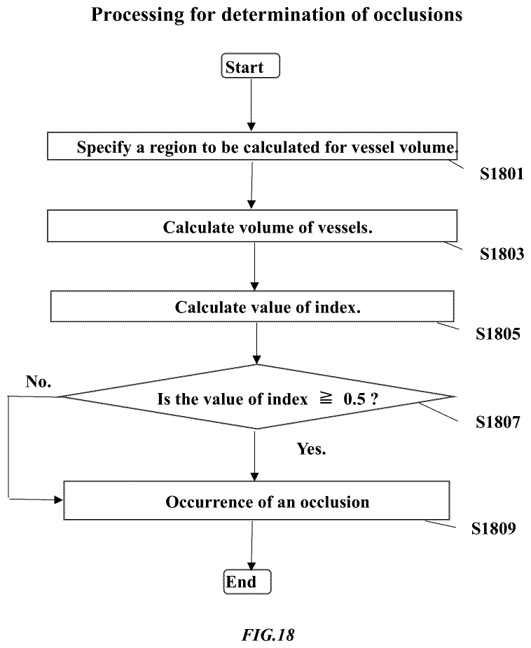

An overview of the processing for determination of occlusions/stenoses is shown in the flowchart of FIG. 17. When the CPU 101a acquires MRA images of the brain from supportive information (S1701), it performs the processing for determination of occlusions (S1703). In addition, the CPU 101a performs the processing for determination of stenoses (S1705).

Thereafter, the CPU 101a uses the results of the processing for determination of occlusions in step S1703 and the processing for determination of stenoses in step S1705 to generate information about occlusions/stenoses (S1707).

(2) The Processing for Determination of Occlusions

As described above, it is difficult to determine directly from MRA images a region where a vascular occlusion has occurred. Thus, a vascular occlusion is detected by utilizing structure of configuration of vessels in the brain, specifically the left-right symmetrical structure, and focusing on the fact that a large difference in blood flow between the left and right side of the brain occurs when a vascular occlusion is occurring.

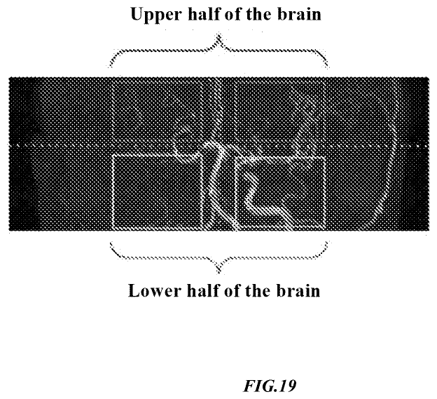

The processing for determination of occlusions is described with reference to the flowchart shown in FIG. 18. The CPU 101a divides an MRA image acquired in step S1801 into four parts including top, bottom, left, and right ones, and specifies a region to be calculated for vessel volume (S1801). Specifically, a predetermined region in MRA image of the brain is divided into four regions of top, bottom, left, and right ones as shown in FIG. 19.

For the detection of occlusions in internal carotid artery (ICA) in the lower half of the region and for the detection of occlusions in the artery after middle cerebral artery (MCA) in the upper half of the region, the brain is divided into upper and lower parts. Besides, because the basilar artery at the center of lower half of the brain may tortuous somehow, a portion of the center is excluded from the regions to be calculated for the volume.

The CPU 101a then calculates the volume of the vessels included in each region (S1803). The CPU 101a calculates the absolute values of the difference in the volume of the vessels in the left and right of the upper side and the left and right of the lower side, respectively, and for normalization, calculates the values divided by the sum of the volumes of the vessels on the left and right sides as a value of index indicating the difference on the left and right sides (S1805). The value of index ranges from 0 to 1, being “0” if there is not any difference on the left and right sides, and being “1” if the vessels on either left or right side completely disappear from MRA images.

When the value of index is “0.5 or higher” (S1807), the CPU 101a determines “occurrence of an occlusion” (S1809).

(3) The Processing for Determination of Stenoses

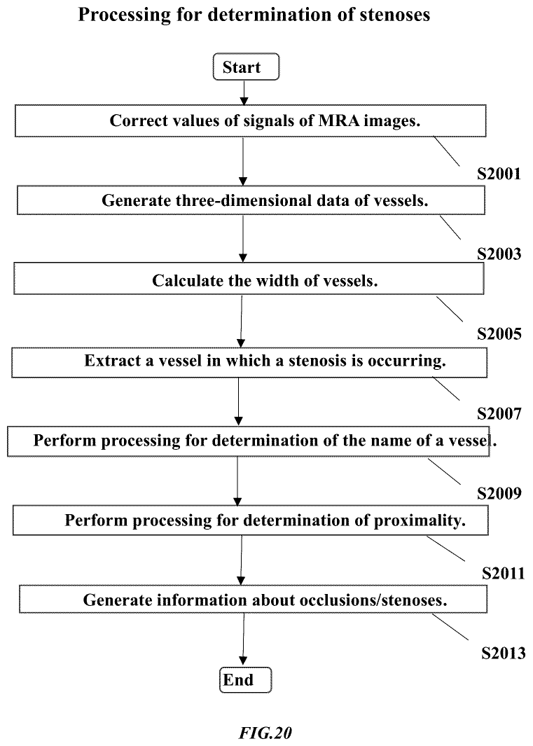

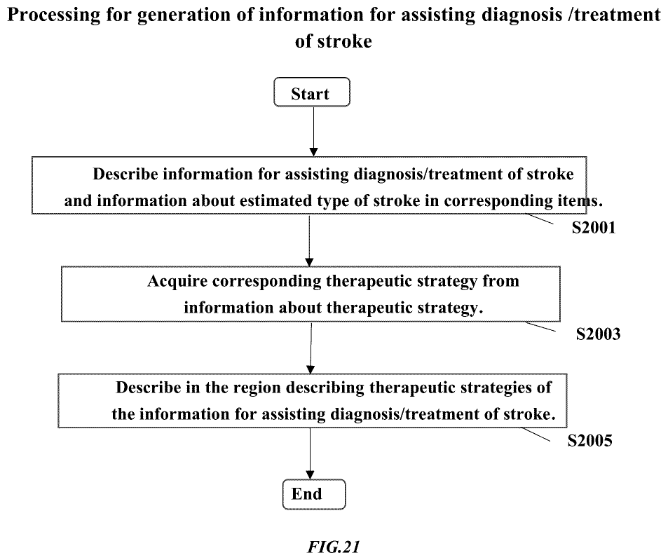

The processing for determination of stenoses is described with reference to the flowchart shown in FIG. 20. The CPU 101a corrects the values of signals of the MRA images acquired in step S1701 (S2001), and then generates the three-dimensional data from which the vessels have been extracted by processing the three-dimensional images (S2003). Since the values of signals of the MRA images are different depending on the model or cases, vessels only cannot be successfully extracted in a binarization processing at a simple threshold. Therefore, correction is performed for the values of the signals of MRA images so that the maximum and the minimum values are the same between the images.

The CPU 101a calculates the width of vessels from three-dimensional data wherein only vessels have been extracted (S2005). Specifically, a centerline of each vessel is estimated from an extracted vessel, and the distance from the centerline to vascular wall is taken as the width (radius) of the vessel.

The CPU 101a extracts a vessel in which the calculated width of a vessel reduces by 50% or more compared with that of surrounding vessels calculated similarly as the vessel in which a stenosis is occurring (S2007).

The CPU 101a uses the three-dimensional data and the width of the vessel extracted in step S2003 to perform a processing for determination of the name of a vessel wherein the name of a vessel is determined (S2009).

In the processing for determination of the name of a vessel, the name of a vessel is determined from the three-dimensional data and the width of the vessel extracted. It is noted that in the processing for determination of the name of a vessel, a model for determination of the name of a vessel trained to calculate the name of a vessel has been constructed in advance by using the three-dimensional data and the width of several vessels.

The CPU 101a uses the information about three-dimensional position in which a stenosis determined in step S2003 is occurring, the stenosed vessels determined in step S2007, and the responsible vessels determined in step S1711 to perform a processing for determination of proximality where if there is an intracranial stenosis related to an infarction is determined (S2011). In the processing for determination of proximality, information about branches of anatomical vessels, such as branching of anterior inferior cerebellar arteries from a basilar artery, is used to determine, for each responsible vessel, the presence or absence of a stenosis in a region beyond the region where an infarction is occurring in the cardiac direction in the responsible vessel.

When the CPU 101a has determined, for a responsible vessel, the presence of a stenosis in a region beyond the region where an infarction is occurring in the cardiac direction in the responsible vessel, it generates information about occlusions/stenoses as “There is an intracranial stenosis related to infarction.” (S2013).

4. The Processing for Determination of the Type of Cerebral Infarction

In the processing for determination of the type of cerebral infarction, Bayesian network is used as algorithm. The Bayesian network has the following characteristics. Results of determination can be acquired as values of probability. Even in cases of lack in some of input items, results of determination can be inferred from other items. The relationship between amounts of characteristic can be visualized.

When considering operations in clinical practice, the second characteristics described above is particularly important. In actual clinical practice, not all test data are available at the time when a determination is desired, and some items are more likely missing. Thus, the Bayesian network, where inference can be performed even in cases of defects, has significant advantages compared to other approaches.

The CPU 101a uses the information about infarction regions generated in step S1517 and the information about occlusions/stenoses generated in step S2013 to determine the type of cerebral infarction, and generates the information about the type of cerebral infarction.

In the processing for determination of the type of cerebral infarction, a plurality of possible names of the type of cerebral infarction paired with their likelihood (%) are generally calculated as the information about the type of cerebral infarction.

It is noted that in the processing for determination of the type of cerebral infarction, a model for determination of the type of cerebral infarction trained to be able to determine the type of cerebral infarction has been constructed in advance by using the information about infarction regions, occlusions/stenoses, and laboratory findings of several cases previously reviewed by physicians. In the process of the constriction of the model for determination of the type of cerebral infarction, contribution rates corresponding to the type of cerebral infarction used in Bayesian network are calculated for the information about infarction regions, occlusions/stenoses, and laboratory findings.

5. The Processing for Generation of Information for Assisting Diagnosis/Treatment of Stroke

The processing for generation of information for assisting diagnosis/treatment of stroke is described with reference to the flowchart shown in FIG. 21. The CPU 101a describes the information about intracranial hemorrhage generated in step S1405 and the information about the type of cerebral infarction generated in step S1311 in corresponding items of the information about determined type of stroke (see FIG. 10) in the information for assisting diagnosis/treatment of stroke (see FIG. 9) generated in step S1303 (S2101).

The CPU 101a acquires from the information about therapeutic strategy (see FIG. 8), the therapeutic strategy corresponding to the information about elapsed time from onset to initiation of treatment of the “non-cardiogenic lacunar infarction”, “non-cardiogenic atherothrombotic cerebral infarction”, and “cardiogenic cerebral embolism” as the items of the region describing estimated type of stroke in the information about determined type of stroke (see FIG. 10) described in the region describing estimated type of stroke of the information for assisting diagnosis/treatment of stroke (see FIG. 9) (S2103), corresponds to the items of the estimated type of stroke, and describes in the region describing therapeutic strategy of the information for assisting diagnosis/treatment of stroke (S2101).

3. Operation (2) of the Device for Providing Information about State of Stroke 103

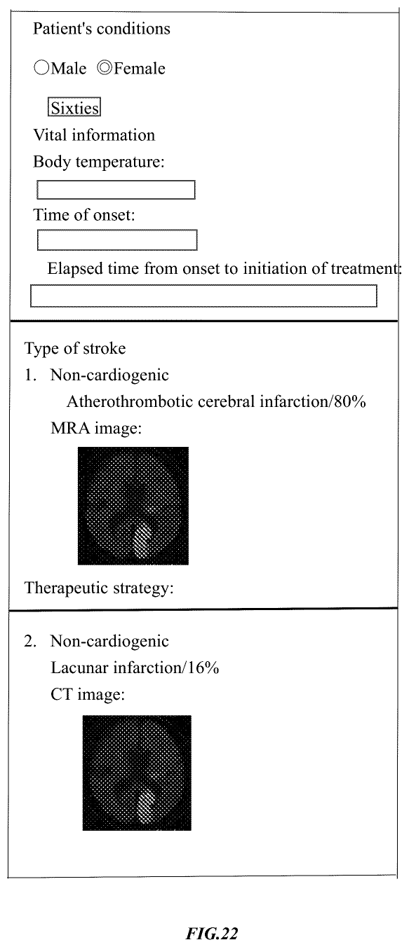

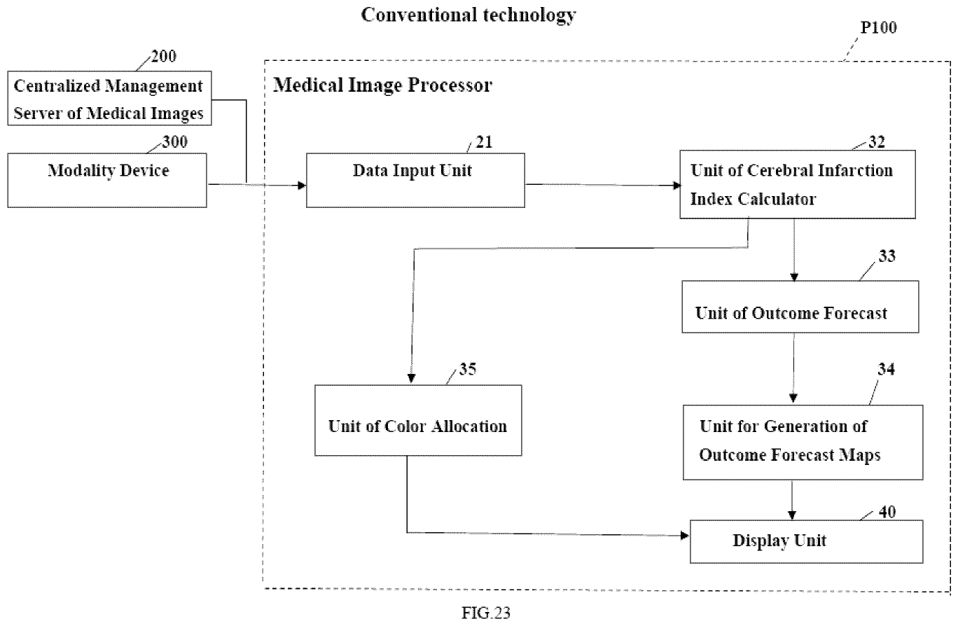

Returning to FIG. 11, when the CPU 103a of the device for providing information about state of stroke 103 has acquired the information for assisting diagnosis/treatment of stroke (S811), it displays the information on the display 103d (S813), as shown in FIG. 22.

When a user of the device for providing information about state of stroke 103 has made a conclusion that the treatment based on the displayed information for assisting diagnosis/treatment of stroke is possible, the necessary treatment is performed.

### OTHER EMBODIMENTS

(1) Sending/Receiving Various Information of Images and Information for Assisting Diagnosis/Treatment of Stroke

In Example 1 described above, various information of images and information for assisting diagnosis/treatment of stroke are sent/received via wireless network between the device for assisting diagnosis/treatment of stroke 101 and the device for providing information about state of stroke 103. However, they may also be sent/received by using wired lines.

(2) Recipients of Information for Assisting Diagnosis/Treatment of Stroke

In Example 1 described above, the information for assisting diagnosis/treatment of stroke is sent to the device for providing information about state of stroke 103 to which each information of images has been sent. However, it may also be sent to other designated communication devices. In addition, it may also be sent to other designated communication devices and the device for providing information about state of stroke 103 to which information of images has been sent.

(3) The Configuration of Hardware of the Device for Assisting Diagnosis/Treatment of Stroke 101

In Example 1 described above, the CPU 101a or the like is used to form the device for assisting diagnosis/treatment of stroke 101. However, it is not limited to the example as long as it can perform the various processing according to the present invention. For example, a dedicated logic circuit may be used to perform various processing on the device for assisting diagnosis/treatment of stroke.

(4) The Configuration of Hardware of the Device for Providing Information about State of Stroke 103

In Example 1 described above, a smartphone is used in the device for providing information about state of stroke 103. However, other devices, such as a dedicated device, may also be used. Furthermore, although the CPU 103a or the like is used to form the device for providing information about state of stroke 103, it is not limited to the example as long as it can perform the various processing according to the present invention. For example, a dedicated logic circuit may be used to perform various processing on the device for providing information about state of stroke.

(5) The Program for Assisting Diagnosis/Treatment of Stroke and the Program for Providing Information about State of Stroke

In Example 1 described above, the program for assisting diagnosis/treatment of stroke achieves the processing according to the illustrated flowchart. However, it is not limited to the example as long as it achieves the similar processing. The same can be said for the program for providing information about state of stroke. Furthermore, the algorithms used in each model is not limited to the example as long as it can exhibit similar functions.

(6) Neurological Findings

Furthermore, in Example 1 described above, neurological findings for presuming classic lacunar syndrome, such as the presence or absence of the clinical evidence such as any of pure motor hemiparesis, pure sensory seizures, ataxic hemiplegia, dysarthria, unilateral dexterity, cerebral cortical infarction (aphasia, agnosia, apraxia, unilateral spatial agnosia, etc.), or cerebellar infarction (dizziness, nausea) may also be used as the information about neurological findings to estimate the type of stroke.

### INDUSTRIAL APPLICABILITY

The system for assisting diagnosis/treatment of stroke according to the present invention can be used, for example, for a medical support system used in a medical institution that does not have any person with professional knowledge about stroke.

### DESCRIPTION OF NOTATIONS

100: system for assisting diagnosis/treatment of stroke
101: device for assisting diagnosis/treatment of stroke
101a: CPU
101b: memory
101c: hard disk drive
101d: keyboard
101e: mouse
101f: display
101g: optical drive
101h: communication circuit
101p: optical media
103: device for providing information about state of stroke
103a: CPU
103b: memory
103f: display
103h: communication circuit

1. A system for assisting diagnosis/treatment of stroke which has a device for assisting diagnosis/treatment of stroke and a device for providing information about state of stroke, uses predetermined information of images that relates to stroke of a subject being diagnosed and information about laboratory findings that indicates the laboratory findings of the subject being diagnosed, to provide information for assisting diagnosis/treatment of stroke that assists diagnosis/treatment of stroke, and is characterized in that
the device for assisting diagnosis/treatment of stroke has
a unit of image acquirer that acquires information of DWI, ADCmap, and MRA images as the information of images,
a unit of laboratory finding acquirer that acquires the information about laboratory findings,
a unit of stroke type determiner that has been trained to use predetermined information of images and predetermined information about laboratory findings to determine information about type of stroke corresponding thereto, and uses the acquired information of images and information about laboratory findings to determine the type of stroke,
a unit of therapeutic strategy acquirer that acquires a corresponding therapeutic strategy from information about therapeutic strategy wherein the therapeutic strategy previously associated with the type of stroke is described, based on the determined type of stroke, and
a unit of information provider for assisting diagnosis/treatment of stroke that provides the determined type of stroke and acquired therapeutic strategy as information for assisting diagnosis/treatment of stroke, and
the device for providing information about state of stroke has
a unit of image provider that provides the information of images,
a unit of information acquirer for assisting diagnosis/treatment of stroke that acquires the information for assisting diagnosis/treatment of stroke, and
a display unit that displays the acquired information for assisting diagnosis/treatment of stroke.

2. A device for assisting diagnosis/treatment of stroke which uses predetermined information of images that relates to stroke of a subject being diagnosed and information about laboratory findings that indicates the laboratory findings of the subject being diagnosed to assist diagnosis/treatment of stroke, and has
a unit of image acquirer that acquires information of DWI, ADCmap, and MRA images as the information of images,
a unit of laboratory finding acquirer that acquires the information about laboratory findings,
a unit of stroke type determiner that has been trained to use predetermined information of images and predetermined information about laboratory findings to determine information about type of stroke corresponding thereto, and uses the acquired information of images and information about laboratory findings to determine the type of stroke,
a unit of therapeutic strategy acquirer that acquires a corresponding therapeutic strategy from information about therapeutic strategy wherein the therapeutic strategy previously associated with the type of stroke is described, based on the determined type of stroke, and
a unit of information provider for assisting diagnosis/treatment of stroke that provides the determined type of stroke and acquired therapeutic strategy as information for assisting diagnosis/treatment of stroke.

3. A device for assisting diagnosis/treatment of stroke characterized in that
furthermore, the unit of stroke type determiner in the device for assisting diagnosis/treatment of stroke according to claim 2 uses the information about infarction regions wherein the size, number, and position of the infarction region, responsible vessels for the infarction region, and their number are determined and the information about occlusions/stenoses wherein the presence or absence of intracranial stenoses is determined, for a cerebral infarction region where a cerebral infarction is occurring, by using the information of DWI, ADCmap, and MRA images, to determine the type of cerebral infarction, and
furthermore, the unit of stroke type determiner has been trained to determine from the predetermined information about infarction regions and the predetermined information about occlusions/stenoses, the type of cerebral infarction corresponding thereto.

4. A device for assisting diagnosis/treatment of stroke characterized in that
furthermore, the unit of stroke type determiner in the device for assisting diagnosis/treatment of stroke according to claim 3 has a unit of cerebral infarction region determiner that uses the information of DWI and ADCmap to determine a cerebral infarction region where a cerebral infarction is occurring, and
the unit of cerebral infarction determiner has been trained to determine from the predetermined information of DWI and ADCmap, the cerebral infarction region corresponding thereto.

5. A device for assisting diagnosis/treatment of stroke characterized in that
furthermore, the unit of stroke type determiner in the device for assisting diagnosis/treatment of stroke according to claim 4 has a unit of responsible vessel determiner that determines responsible vessels from the information about three-dimensional position of the predetermined cerebral infarction regions, and
the unit of responsible vessel determiner has been trained to be able to determine from the information about three-dimensional position of the predetermined cerebral infarction regions, the responsible vessels corresponding thereto.

6. A program for assisting diagnosis/treatment of stroke that makes a computer function in the device for assisting diagnosis/treatment of stroke that uses predetermined information of images that relates to stroke of a subject being diagnosed and information about laboratory findings that indicates the laboratory findings of the subject being diagnosed, to assist diagnosis/treatment of stroke, as a unit of image acquirer that acquires information of DWI, ADCmap, and MRA images as the information of images,
a unit of laboratory finding acquirer that acquires the information about laboratory findings,
a unit of stroke type determiner that has been trained to use predetermined information of images and predetermined information about laboratory findings to determine information about type of stroke corresponding thereto, and uses the acquired information of images and information about laboratory findings to determine the type of stroke,
a unit of therapeutic strategy acquirer that acquires a corresponding therapeutic strategy from information about therapeutic strategy wherein the therapeutic strategy previously associated with the type of stroke is described, based on the determined type of stroke, and
a unit of information provider for assisting diagnosis/treatment of stroke that provides the determined type of stroke and acquired therapeutic strategy as information for assisting diagnosis/treatment of stroke.

7. A model for determination of the type of cerebral infarction, characterized by making a computer function as a unit of stroke type determiner that uses the information about infarction regions wherein the size, number, and position of the infarction region, responsible vessels for the infarction region, and their number are determined and the information about occlusions/stenoses wherein the presence or absence of intracranial stenoses is determined, for a cerebral infarction region where a cerebral infarction is occurring, by using the information of DWI, ADCmap, and MRA images, to determine the type of cerebral infarction, and that
furthermore, the unit of stroke type determiner has been trained to determine from the predetermined information about infarction regions and the predetermined information about occlusions/stenoses, the type of cerebral infarction corresponding thereto.

8. A model for determination of cerebral infarction regions, characterized by making a computer function as a unit of image acquirer that acquires information of DWI and ADCmap of the brain and a unit of cerebral infarction region determiner that determines a cerebral infarction region where a cerebral infarction is occurring by using the information of DWI and ADCmap, and that
the unit of cerebral infarction determiner has been trained to determine from the predetermined information of DWI and ADCmap, a cerebral infarction region corresponding thereto.

9. A model for determination of responsible vessels, characterized by making a computer function as a unit of responsible vessel determiner that determines responsible vessels from the information about three-dimensional position of a cerebral infarction region where a cerebral infarction is occurring, and that
the unit of responsible vessel determiner has been trained to be able to determine from the predetermined information about three-dimensional position of the cerebral infarction regions, the responsible vessels corresponding thereto.
